(19) United States
(12) Patent Application Publication
(10) Pub. No.: US 2005/0176005 A1
(43) Pub. Date: Aug. 11, 2005

(54) Functional ligand display

(76) Inventor: Matthew A. Spear, Enclitas, CA (US)

Correspondence Address: Maha A Hamdan;Medlen & Carroll, Suite 350, 101 Howard Street, San Francisco, CA, 94105, US

(21) Appl. No.: 10/501,609

(22) PCT Filed: Jan. 16, 2003
(86) PCT No.: PCT/US03/01426

(30) Foreign Application Priority Data
Jan. 16, 2002 (US) 60349893

(51) Int. Cl. C12Q 1/68; G01N 33/574; C12N 5/08

(52) U.S. Cl. 435/6; 435/7.23; 435/372

(57) ABSTRACT

The present invention relates to methods involving the selection of peptides from complex displayed libraries to induce or detect signal transduction pathway activation. In one embodiment, the present invention provides methods for the selection of peptides capable of binding a viable cell and subsequently triggering or detecting programmed cell death (e.g., apoptosis). The present invention also provides compositions comprising the selected peptides suitable for medicinal and research use.

### FIELD OF THE INVENTION

The present invention relates to methods involving the selection of peptides from complex displayed libraries to induce or detect signal transduction pathway activation. In one embodiment, the present invention provides methods for the selection of peptides capable of binding a viable cell and subsequently triggering or detecting programmed cell death (e.g., apoptosis). The present invention also provides compositions comprising the selected peptides suitable for medicinal and research use.

### BACKGROUND OF THE INVENTION

It is known that numerous cell surface receptors exist, the activation of which can induce apoptosis (e.g., programmed cell death), or other signal transduction pathways responsible for cell death, survival, growth, replication, differentiation, etc. Some receptors are selectively expressed on certain cell types including tumor cells. Much research in molecular biology is devoted to identifying these receptors, and elucidating their biology. This knowledge in turn, is used in the development of medicines to treat a broad spectrum of illnesses, including but not limited to cancer, autoimmune disease, cardiovascular disease and Alzheimer's disease. Methods for identifying cell surface receptors frequently entail painstaking elucidation of the function of genes or proteins that have been found serendipitously by a variety of means. Once a receptor pathway has been identified, ligands or other interacting molecules can then be developed as therapeutic or diagnostic agents. However, some cell surface receptors do not have known or useable naturally-occurring, selective ligands and thus monoclonal antibodies (MAb) are often used to specifically target them. Unfortunately, even in the most truncated forms, MAb are large molecules that have limited biodistributions and present significant potential problems for delivery. Subsequent to the discovery and characterization of receptor pathways, small molecule therapeutics can also be developed through extensive screening or directed design. However, these methods are cumbersome and time-consuming.

Thus, there is a need in the art for methods to quickly and efficiently identify small molecules, which have the ability to modulate cellular signal transduction pathways such as apoptosis. The need is particularly acute in cases where large libraries are involved. The small molecules or peptides identified in this way are contemplated to be suitable for use as therapeutic and/or diagnostic agents, as well as research reagents.

### SUMMARY OF THE INVENTION

The present invention relates to methods involving the selection of peptides from complex displayed libraries to induce or detect signal transduction pathway activation. In one embodiment, the present invention provides methods for the selection of peptides capable of binding a viable cell and subsequently triggering or detecting programmed cell death (e.g., apoptosis). The present invention also provides compositions comprising the selected peptides suitable for medicinal and research use.

In some embodiments, the present invention provides methods for selecting a ligand from a library comprising the steps of providing target cells, a library comprising a ligand capable of binding to and activating the target cells, an indicator, and an isolation means; contacting the target cells with the library, under conditions suitable to affect a cellular response in a subset of the target cells thereby yielding at least one activated target cell; exposing the target cells having been contacted with the library to the indicator, under conditions such that the at least one activated target cell is detected; collecting the at least one activated target cell having been detected with the indicator by use of the isolation means; and recovering the ligand from the at least one activated target cell having been collected by use of the isolation means. In some preferred embodiments, the library is a phage display library. In other preferred embodiments at least one target cell is a cancer cell. In related embodiments, the cancer cell is an acute lymphoblastic leukemia cell, while in particularly preferred embodiments, the acute lymphoblastic leukemia cell is selected from the group consisting of a JURKAT cell, a MOLT-4 cell, a TALL-104 cell and a patient ALL cell. In some embodiments, the cellular response comprises a response selected from the group consisting of apoptosis, proliferation, differentiation, adhesion, migration, cytokine secretion, and cessation of such processes. The invention also provides embodiments in which the cellular response comprises a response selected from the group consisting of phosphorylation, dephosphorylation, calcium flux, target molecule cleavage, protein-protein interaction, protein-nucleic acid interaction, nucleic acid-nucleic acid interaction, and production of detectable fluorescence. In some embodiments in which the cellular response comprises apoptosis, the indicator comprises fluorescent-labeled Annexin V. In preferred embodiments, the isolation means comprises fluorescence activated cell sorting, while in other embodiments, the isolation means comprises a magnet. In addition, in some preferred embodiments, the ligand selected by the disclosed method is provided.

In other embodiments, the present invention also provides methods for selecting a ligand from a library comprising the steps of providing target cells comprising at least one activated target cell, a library comprising a ligand capable of binding to the at least one activated target cell, an indicator, and an isolation means; contacting the target cells with the library, under conditions suitable to affect binding of the ligand to the at least one activated target cell; exposing the target cells having been contacted with the library to the indicator, under conditions such that the at least one activated target cell is detected; collecting the at least one activated target cell having been detected with the indicator by use of the isolation means; and recovering the ligand from the at least one activated target cell having been collected by use of the isolation means. In some preferred embodiments, the library is a phage display library. In other preferred embodiments the at least one target cell is a cancer cell. In related embodiments, the cancer cell is an acute lymphoblastic leukemia cell, while in particularly preferred embodiments, the acute lymphoblastic leukemia cell is selected from the group consisting of a JURKAT cell, a MOLT-4 cell, a TALL-104 cell and a patient ALL cell. In some embodiments, the at least one activated target cell is undergoing a cellular response comprising a response selected from the group consisting of apoptosis, proliferation, differentiation, adhesion, migration, cytokine secretion, and cessation of such processes. The invention also provides embodiments in which the cellular response comprises a response selected from the group consisting of phosphorylation, dephosphorylation, calcium flux, target molecule cleavage, protein-protein interaction, protein-nucleic acid interaction, nucleic acid-nucleic acid interaction, and production of detectable fluorescence. In some embodiments in which the cellular response comprises apoptosis, the indicator comprises fluorescent-labeled Annexin V. In preferred embodiments, the isolation means comprises fluorescence activated cell sorting, while in other embodiments, the isolation means comprises a magnet. In addition, in some preferred embodiments the ligand selected by the disclosed method is provided.

The present invention contemplates in one embodiment a method, comprising: a) providing; i) target cells, ii) a library comprising a plurality of ligands, wherein at least one ligand is capable of binding so as to cause a response from at least a subset of said target cells, and iii) an indicator; b) contacting the target cells with said ligands of said library to create treated target cells, under conditions such that a subset of said treated target cells is activated; c) exposing said treated target cells to said indicator, under conditions such that the at least one activated target cell is detected to create a detected activated target cell; d) collecting said detected activated target cell to create a collected activated target cell; and e) recovering said ligand from said collected activated target cell. In another embodiment, the present invention contemplates that the library is a phage display library. In yet another embodiment, the present invention contemplates that the target cell is a cancer cell. In still yet another embodiment, the present invention contemplates that the cancer cell is an acute lymphoblastic leukemia cell. In still yet another embodiment, the present invention contemplates that the acute lymphoblastic leukemia cell is selected from the group consisting of a JURKAT cell, a MOLT-4 cell, a TALL-104 cell and a patient ALL cell. In still yet another embodiment, the present invention contemplates that the cellular response comprises a response selected from the group consisting of apoptosis, proliferation, differentiation, adhesion, migration, cytokine secretion, and cessation of such said processes. In still yet another embodiment, the present invention contemplates that the cellular response comprises a response selected from the group consisting of phosphorylation, dephosphorylation, calcium flux, target molecule cleavage, protein-protein interaction, protein-nucleic acid interaction, nucleic acid-nucleic acid interaction, and production of detectable fluorescence. In still yet another embodiment, the present invention contemplates that the indicator comprises fluorescent-labeled Annexin V.

The present invention contemplates that in one embodiment, a method, comprising: a) providing; i) a library comprising a ligand capable of binding to at least one activated target cell, ii) an indicator, and, iv) an isolation means; b) contacting the target cells with the library, under conditions suitable to affect binding of the ligand to the at least one activated target cell; c) exposing the target cells having been contacted with the library to the indicator, under conditions such that the at least one activated target cell is detected; d) collecting the at least one activated target cell having been detected with the indicator by use of the isolation means, and; e) recovering the ligand from the at least one activated target cell having been collected by use of the isolation means. In another embodiment, the present invention contemplates that the library is a phage display library. In yet another embodiment, the present invention contemplates that the target cell is a cancer cell. In still yet another embodiment, the present invention contemplates that the cancer cell is an acute lymphoblastic leukemia cell. In still yet another embodiment, the present invention contemplates that the acute lymphoblastic leukemia cell is selected from the group consisting of a JURKAT cell, a MOLT-4 cell, a TALL-104 cell and a patient ALL cell. In still yet another embodiment, the present invention contemplates that the cellular response comprises a response selected from the group consisting of apoptosis, proliferation, differentiation, adhesion, migration, cytokine secretion, and cessation of such said processes. In still yet another embodiment, the present invention contemplates that the cellular response comprises a response selected from the group consisting of phosphorylation, dephosphorylation, calcium flux, target molecule cleavage, protein-protein interaction, protein-nucleic acid interaction, nucleic acid-nucleic acid interaction, and production of detectable fluorescence. In still yet another embodiment, the present invention contemplates that the indicator comprises fluorescent-labeled Annexin V.

### DESCRIPTION OF THE FIGURES

The following Figures form part of the Specification and are included to further demonstrate certain aspects and embodiments of the present invention. The invention may be better understood by reference to one or more of these Figures in combination with the detailed description of specific embodiments presented therein.

FIG. 1 illustrates the biopanning method of targeted phage selection from a phage display library. This method entails the incubation of a phage expressing random coat protein sequences on a fixed target. Phage which do not bind to the target are washed away, while bound phage are eluted and regrown. These steps are repeated to enrich the desired phage.

FIG. 2 shows the results obtained from a viable solid phase ELISA used to quantitate targeted CMTI#2 phage relative to untargeted control phage binding to U87-MG malignant glioma cells, and to control cell lines 9L and PANC-1.

### DESCRIPTION OF THE INVENTION

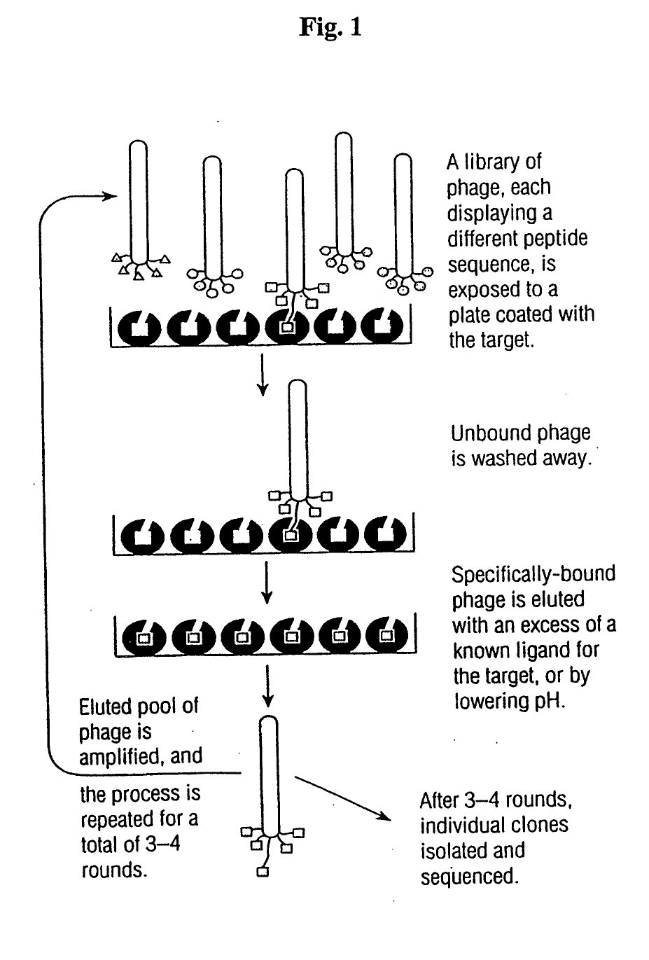

Phage display can provide a source of small peptide ligand epitopes for binding cellular receptors. Early methods involving this technique (WO 90/02809 [1990]; and Parmley and Smith, Gene 73: 305-318 [1989]), initially utilized nearly random oligonucleotide sequences inserted into the filament binding protein of a bacterial phage (frequently protein III of the M13 E. coli phage). In this way, a phage library expressing approximately 10⁷-10⁹ different peptides can be generated. MAb variable regions and other moieties have also been used in place of small peptides. Phage expressing a peptide sequence that by chance has affinity for a specific molecule or tissue that has been fixed to a substrate, can then be selected for expansion by sequential binding and elution steps (See, FIG. 1). The DNA sequences encoding the bound peptides are subsequently recovered from the phage genome.

Phage display peptides that target a number of molecules, cellular proteins and tissues, have been reported (O'Neil and Hoess, Current Opinion in Structural Biology 5: 443-449 [1995]; and Pasqualini and Ruoslahti, Nature 380: 364-366 [1996]). More recently, peptide epitopes selected by phage display have been used to target pharmaceuticals to tumors (Arap et al., Science 279: 377-380 [1998]). Small peptides that selectively bind tumor cells or tumor neovasculature additionally have the potential to serve as radiolabeled Or contrast-tagged imaging agents, as has been demonstrated with ¹¹¹In labeled octreotide (Hallenbeck et al., Human Gene Therapy 10: 1721-1733 [1999]). This methodology has significant potential utility because uncertainty frequently exists in the diagnostic imaging of suspected tumor recurrences and in the radiographic evaluation of primary and recurrent tumor spread.

The ligand identification via expression (LIVE) selection strategy is based on the principle of ligand-conferred cell tropism. The expression of an appropriate ligand on the surface of phage has been reported to confer mammalian cell tropism to the resulting phage particles, with subsequent cell binding, uptake and expression of genes carried by the phage (Larocca et al., Human Gene Therapy 9: 2393-2399 [1998]; and Larocca et al., FASEB Journal 13: 727-734 [1999]). Phage-transduced cells can then be isolated via fluorescence activated cell sorting (FACS). To perform the LIVE selection a phage library is created using a vector that carries a green fluorescent protein (GFP) expression cassette driven by a cytomegalovirus promoter. The target cells are incubated with the phage library and GFP-positive cells are subsequently isolated by FACS. The phage genome from the transduced cells is then recovered and cloned, revealing the targeting peptide sequence.

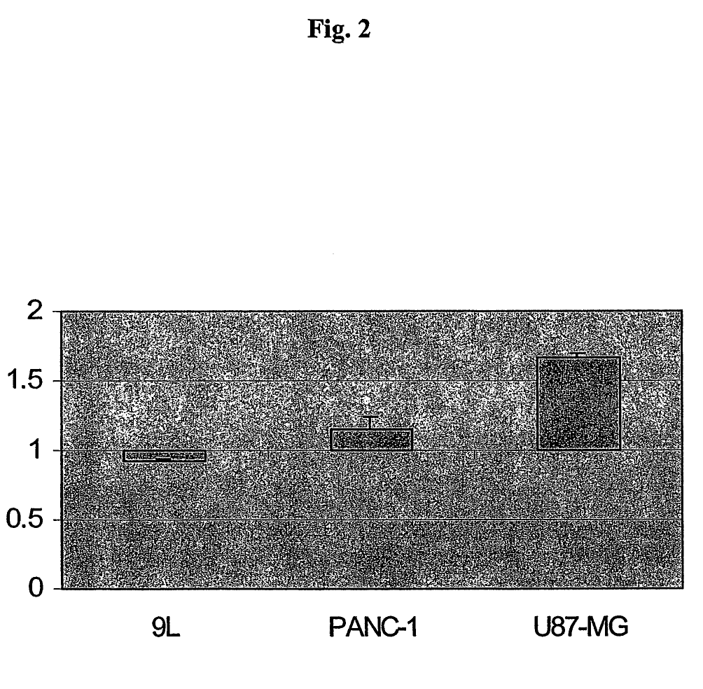

Phage have been successfully selected from libraries with two different epitope repertoires, TN10 and CMTI, against cell surface receptors on viable U87-MG glioma cells by using a modified biopanning plus centrifugation technique to recover cell bound phage (Spear et al., Cancer Gene Therapy 8: 506-511 [2001]). The epitope variable regions of recovered phage were then expanded and sequenced. The CMTI library, initially containing phage expressing 2×10⁷ different epitope sequences, collapsed after four rounds of selection such that 42% of recovered clones expressed a consensus sequence. Selective binding to viable adherent U87-MG cells was demonstrated under physiologic conditions to be 167% (+/−27%) that of unselected phage using a novel viable enzyme linked immunosorbent assay (ELISA). In comparison, there was no difference in binding between the selected and unselected phage to control 9L rat gliosarcoma cells, and to PANC-1 human pancreatic adenocarcinoma cells (See, FIG. 2). By using PCR, the epitope was recovered with unique flanking restriction sites for insertion into an appropriate vector.

A novel functional ligand display (FLD) method has subsequently been developed to expand phage bearing peptides that either induce apoptosis or bind to apoptotic markers on the cell surface. It is contemplated that phage, each carrying multiple copies of the expressed small peptide, not only bind to cell surface receptors as previously demonstrated (See, FIG. 2), but activate the receptors and downstream signal transduction pathways. Receptor activation may occur through cross linking or through conformation-induced changes. However, an understanding of the mechanism(s) is not necessary in order to make and/or use the present invention.

Alternatively, the FLD method provides a means to identify phage which bind to receptors expressed after induction of apoptosis, thus uncovering new markers of apoptosis. For instance, after incubation with a T7 phage display library, apoptotic fractions of JURKAT cells were selected by FITC-Annexin V staining and cell sorting, thereby permitting the selective recovery of phage bound to cells undergoing apoptosis. The isolated small peptides are contemplated to have therapeutic and/or diagnostic use in refractory acute lymphoblastic leukemia (ALL). In addition, characterization and quantification of the complementary receptors uncovered with this novel proteomics method, is contemplated to improve understanding of the biology of ALL, as well as that of other malignancies.

Definitions

To facilitate an understanding of the present invention, a number of terms and phrases are defined below:

The term “selecting” as used herein, refers to the process whereby a phenotypic characteristic is used to enrich a population for those individuals displaying the desired phenotype.

As used herein the term “ligand” refers to a molecule, such as a random peptide or variable segment sequence, with the affinity to bind to a second molecule or receptor. As one of skill in the art will recognize, a molecule can be both a receptor and a ligand.

The term “library” as used herein, refers to a collection of nucleic acid or amino acid sequences, and especially recombinant DNA or synthetic peptides that are maintained in an appropriate environment.

As used herein, the terms “bacteriophage” and “phage” refer to a bacterial virus containing a DNA core and a protective proteinaceous shell.

The term “phage-display library” refers to a protein expression library, constructed in bacteriophage vector, that expresses a collection of protein sequences as fusions with a phage coat protein. Thus, in the context of the invention, single-chain recombinant proteins having ligand-binding potential are expressed as fusion proteins on the exterior of the phage particle. This combination advantageously allows contact and binding between the recombinant binding protein and an immobilized ligand. Those having ordinary skill in the art will recognize that phage clones expressing binding proteins specific for the ligand can be substantially enriched by serial rounds of phage binding to the immobilized ligand, dissociation from the immobilized ligand and, amplification by growth in bacterial host cells. The present invention is not limited to a phage display library. Other amino acid, protein or peptide libraries are also contemplated for use with the present invention.

As used herein the term “target cells,” refers to the cell population expressing a receptor(s) for which a ligand is desired. Preferred “target cells” include but are not limited to “cancer cells.” “Cancer cells” refer to cells capable of endless replication, which may also have the properties of loss of contact inhibition, invasiveness and the ability to metastasize. Particularly preferred “cancer cells” are “acute lymphoblastic leukemia cells” or “ALL cells.” Suitable ALL cells include without limitation cell lines, as well as cancer cells obtained from patients with a lymphocytic leukemia marked by an abnormal increase in the number of lymphoblasts and characterized by a rapid onset or progression of symptoms which include several of fever, anemia, pallor, fatigue, appetite loss, joint pain, bleeding, thrombocytopenia, granulocytopenia, enlargement of the lymph nodes, liver and spleen.

The term “binding to” as used herein, refers to the process of combining or interacting by chemical forces.

As used herein, the term “activating” refers to the process of initiating a response from a cell.

The term “cellular response” refers to the gain or loss of activity by a cell. For example, the “cellular response” may constitute but is not limited to apoptosis, proliferation, differentiation, adhesion, migration and/or cytokine secretion, or cessation of such processes. The terms “apoptosis” and “programmed cell death” refer to a type of cell death characterized by DNA fragmentation and membrane blebbing, which is distinct from necrosis. The term “proliferation” refers to an increase in cell number. The term “differentation” refers to the maturation process cells undergo whereby they develop distinctive characteristics, or perform specific functions, and are less likely to divide. The term “adhesion” refers to the attachment of cells to other cells or to the extracellular matrix. As used herein, the terms “migration” or “chemotaxis” refer to the movement of cells in which the direction of movement is affected by the gradient of a diffusible substance. The term “cytokine section” refers to the release of a cytokine from a cell.

In the context of the invention, the “cellular response” may comprise phosphorylation, dephosphorylation, calcium flux, target molecule cleavage, protein-protein interaction, protein/nucleic acid interaction, nucleic acid-nucleic acid interaction, and/or the production of detectable fluorescence. The term “phosphorylation” refers to the addition of phosphate groups. Protein phosphorylation is catalyzed by protein kinases which attach phosphate groups to hydroxyls of Ser, Thr or Tyr side chains. The term “dephosphorylation” refers to the removal of a phosphate group. Protein dephosphorylation is catalyzed by protein phosphatases which remove phosphate groups from the side chains of Ser, Thr, or Tyr. The term “calcium flux” refers to the mobilization of calcium either as an influx from the extracellular medium or from the release of intracellular stores. As used herein, the term target molecule cleavage refers to the splitting of a molecule. The term “interaction” refers to the reciprocal action or influence of two or more molecules.

As used herein, the term “indicator” refers to a substance used to show the condition of a system with respect to the presence or activity of a particular material. In the context of the invention, the indicator reveals whether a cellular response has occurred. Preferred indicators include but are not limited to annexin V, monoclonal antibodies, fluorescent nucleotide probes, and Indo-1.

The term “annexin V” refers to a calcium-dependent phospholipid-binding protein which has a high affinity for phospholipid serine. In the context of the invention, annexin V is used as a marker for dying cells. During the early stages of apoptosis, cell membranes lose their phospholipid symmetry and expose phosphatidylserine at the cell surface. Necrotic cells by virtue of loss of membrane integrity also expose phosphatidylserine, although unlike apoptotic cells, necrotic cells can be stained with propidium iodide. The term “propidium iodide” refers to a membrane-impermeant dye that stains by intercalating into nucleic acid molecules.

As used herein, the term “isolation means” refers to a device, apparatus or technique for separating out individuals from a population (e.g., cells). Preferred “isolation means” include but are not limited to a FACS or a magnet.

The terms “fluorescent activated cell sorter” and “FACS” refer to a device for sorting particles according to their fluorescence and light scattering properties. In the context of the invention, cells marked with a fluorescent label are sorted based upon how much they fluoresce at a particular wavelength.

The term “magnet” as used herein, refers to a body having the property of attracting iron and producing a magnetic field external to itself.

The term “subset” as used herein, refers to a portion of a population or group. The subset may have, but need not have, a characteristic that distinguishes it from the population or group.

The terms “activated cells” and “responded cells” as used herein, are synonymous and refer to cells that have had, for example, cellular physiological or chemical processes initiated, modified or inhibited to some degree by the reversible or irreversible binding of, for example, a ligand.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ALL (acute lymphoblastic leukemia); FACS (fluorescence activated cell sorter); FITC (fluorescein isothiocyanate); FLD (functional ligand display); MAb (monoclonal antibody); ° C. (degrees Centigrade); RT (room temperature); FBS (fetal bovine serum); PBS (phosphate-buffered saline); bp (base pair); kb (kilobase pair); kD (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); MW (molecular weight); sec (seconds); min(s) (minute/minutes); hr(s) (hour/hours); PCR (polymerase chain reaction); Dyax (Dyax Corporation, Cambridge, Mass.); New England Biolabs (New England Biolabs, Inc., Beverly, Mass.); Novagen (Novagen, Inc., Madison, Wis.); and Pharmingen (BD Biosciences, Pharmingen, San Diego, Calif.).

### Example 1

### Selection of Apoptosis-Inducing Phage

To clone phage inducing apoptosis, supernatant containing approximately 1×10⁹ unbound phage from a phage display library was incubated with 10⁶ JURKAT cells in 5 ml RPMI/FBS at 37° C. for 2 hrs. Cells were pelleted at 1200 rpm for 2 min, then washed in 2×PBS. Washed cells were resuspended in a solution containing fluorescein isothiocyanate (FITC)-labeled Annexin V (Pharmingen) at 3×10⁶ cells/ml. Annexin V selectively binds to phosphatidyl serine moieties that have been translocated to the outer plasma membrane of the cell, as an early apoptotic event (Vermes et al., Journal of Immunological Methods 184: 39-51 [1995]). Cells were incubated with FITC-Annexin V at RT in the dark for 15 min. FACS was performed within 1 hour with gating (excitation=488 nm, emmission=520 nm, Annexin V positive, propidium iodide negative) to sort apoptotic cells. Phage bound to these cells or internalized were recovered and expanded through direct incubation with Escherichia coli. Selection was repeated four times with the percentage of apoptotic cells being compared to the previous selection and to cells incubated with unselected phage.

An increasing percentage of apoptotic cells is considered a positive result and selection rounds continue until the apoptotic cell percentage plateaus. Individual selected phage colonies are then recovered, expanded and the epitope variable region is sequenced. Sequences that appear more than three times in those recovered are indicative of specific induction of apoptosis and a collapsed phage library.

In the event that increased apoptosis is also seen upon incubation with control lymphocytes, alternating negative selection rounds with normal lymphocytes may be necessary to select out epitopes which bind to noncancerous cells. An initial subtractive selection is attempted against normal human cells (e.g., lymphocytes or fibroblasts) that would not be expected to display tumor associated antigens, in order to remove non-specific human cell binding ligands from the library. This step is contemplated to increase the probability of obtaining tumor targeting ligands specifically directed to ALL.

Alternating ALL cell lines (MOLT-4 and TALL-104) and patient cancer specimens in selection rounds, is also done to increase the probability of selecting for phage which bind to receptors expressed on a broad range of ALL cells. It is possible that through negative selection, positive clones are removed that are weak binders and that are present in low abundance in the library. For this reason, parallel rounds of selection are done with a library that is subjected to negative selection and a library that is not.

The FLD method described above is also contemplated to be used to select phage selectively binding to cells already undergoing apoptosis. In this way, the method provides a means of discovering new cell surface receptors that are markers of apoptosis. Likewise, this novel proteomics method is contemplated to be used to select phage selectively binding to cells in which other signal transduction pathways have been triggered, by using markers such as transcription factor-reactive MAbs or enzymatic substrates, instead of Annexin V.

Various types of phage display libraries find use in the present invention. For instance, CMTI (Dyax) or Ph.D-12 (New England Biolabs) libraries are appropriate, as is the T7Select system (Novagen). Other recoverable systems carrying different types of displayed ligands, such as synthetic organic molecules or DNA, also find use in the FLD method.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as should not be unduly limited to such specific embodiments. Indeed, various modifications of the described modes for carrying out the invention which are obvious to those skilled in the art and/or related fields are intended to be within the scope of the present invention.

1. A method, comprising:
a) providing; i) target cells, ii) a library comprising a plurality of ligands, wherein at least one ligand is capable of binding so as to cause a response from at least a subset of said target cells, and iii) an indicator;
b) contacting the target cells with said ligands of said library to create treated target cells, under conditions such that a subset of said treated target cells is activated;
c) exposing said treated target cells to said indicator, under conditions such that the at least one activated target cell is detected to create a detected activated target cell;
d) collecting said detected activated target cell to create a collected activated target cell; and
e) recovering said ligand from said collected activated target cell.

2. The method of claim 1, wherein said library is a phage display library.

3. The method of claim 1, wherein said target cell is a cancer cell.

4. The target cell of claim 3, wherein said cancer cell is an acute lymphoblastic leukemia cell.

5. The acute lymphoblastic leukemia cell of claim 4, wherein said cell is selected from the group consisting of a JURKAT cell, a MOLT-4 cell, a TALL-104 cell and a patient ALL cell.

6. The method of claim 1, wherein said cellular response comprises a response selected from the group consisting of apoptosis, proliferation, differentiation, adhesion, migration, cytokine secretion, and cessation of such said processes.

7. The method of claim 1, wherein said cellular response comprises a response selected from the group consisting of phosphorylation, dephosphorylation, calcium flux, target molecule cleavage, protein-protein interaction, protein-nucleic acid interaction, nucleic acid-nucleic acid interaction, and production of detectable fluorescence.

8. The method of claim 1, wherein said indicator comprises fluorescent-labeled Annexin V.

9. A method, comprising:
a) providing; i) a library comprising a ligand capable of binding to at least one activated target cell, ii) an indicator, and, iv) an isolation means;
b) contacting the target cells with the library, under conditions suitable to affect binding of the ligand to the at least one activated target cell;
c) exposing the target cells having been contacted with the library to the indicator, under conditions such that the at least one activated target cell is detected;
d) collecting the at least one activated target cell having been detected with the indicator by use of the isolation means, and;
e) recovering the ligand from the at least one activated target cell having been collected by use of the isolation means.

10. The method of claim 9, wherein said library is a phage display library.

11. The method of claim 9, wherein said target cell is a cancer cell.

12. The target cell of claim 11, wherein said cancer cell is an acute lymphoblastic leukemia cell.

13. The acute lymphoblastic leukemia cell of claim 12, wherein said cell is selected from the group consisting of a JURKAT cell, a MOLT-4 cell, a TALL-104 cell and a patient ALL cell.

14. The method of claim 9, wherein said cellular response comprises a response selected from the group consisting of apoptosis, proliferation, differentiation, adhesion, migration, cytokine secretion, and cessation of such said processes.

15. The method of claim 9, wherein said cellular response comprises a response selected from the group consisting of phosphorylation, dephosphorylation, calcium flux, target molecule cleavage, protein-protein interaction, protein-nucleic acid interaction, nucleic acid-nucleic acid interaction, and production of detectable fluorescence.

16. The method of claim 9, wherein said indicator comprises fluorescent-labeled Annexin V.
